(19) United States
(12) Patent Application Publication
(10) Pub. No.: US 2004/0029181 A1
(43) Pub. Date: Feb. 12, 2004

(54) Novel metal complex and method for determining amino acid sequence of protein using the same

(76) Inventors: Norikazu Ueyama, Osaka (JP); Taka-Aki Okamura, Osaka (JP); Shigemi Norioka, Osaka (JP); Takashi Nakazawa, Nara-shi (JP); Hiroki Kuyama, Kyoto-shi (JP); Eiji Ando, Kyoto-shi (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/614,324

(22) Filed: Jul. 8, 2003

(30) Foreign Application Priority Data
Jul. 12, 2002 (JP) 2002-204864

(51) Int. Cl. G01N 33/53; C07K 14/00

(52) U.S. Cl. 435/7.1; 530/400

(57) ABSTRACT

The present invention provides a novel metal complex useful for a reagent for determining the amino acid sequence of protein or peptide, and a method of using it for determining the amino acid sequence of protein or peptide. The metal complex which has a functional group capable of forming a covalent bond with the amino group of the N-terminal amino acid residue of protein or peptide or with the carboxyl group of the C-terminal amino acid residue of protein or peptide. In general, the functional group is in the ligand of the complex. The metal complex is reacted with a protein or peptide (A) of which the amino acid sequence is to be determined, to thereby obtain a metal complex derivative (B) where the covalent bond of the functional group of the metal complex with the amino group of the N-terminal amino acid residue of the protein or peptide (A) or with the carboxyl group of the C-terminal amino acid residue of protein or peptide is formed, and the derivative (B) is analyzed through mass spectrometry to determine the amino acid sequence of the protein or peptide (A).

### BACKGROUND OF THE INVENTION

1. Field of the Invention

The present invention relates to a novel metal complex useful for a reagent for determining the amino acid sequence of protein or peptide, and to a method of using it for determining the amino acid sequence of protein or peptide.

2. Disclosure of the Related Art

An Edman method is popular for determining the N-terminal amino acid sequence of protein, which comprises the steps of labeling the N-terminal amino acid of protein with phenyl isothiocyanate (PITC), eliminating the N-terminal amino acid to be a 2-anilino-5-thiazolinone derivative, converting it into a 3-phenylthiohydantoin derivative (PTH-amino acid), and analyzing the final product, 3-phenylthiohydantoin derivative through high-performance liquid chromatography (HPLC) to thereby identify the amino acid. Analyzing the phenylthiohydantoin derivative of amino acid through HPLC is effected generally through UV absorption at 269 nm, but the absorption coefficient is small and the detection sensitivity is low. In addition, the Edman reaction itself takes a lot of time.

A modified Edman method has been reported, which comprises labeling the N-terminal amino acid of a protein with a fluorescent substance such as fluorescein isothiocyanate but not with PITC to determine the N-terminal amino acid sequence of the protein. However, this method is also problematic in that the detection sensitivity is low since the fluorescent intensity of the final product obtained after the labeling with the fluorescent substance is low, and the stability of the final product is poor.

On the other hand, de novo sequencing through LCMS tandem mass spectrometry (CID) and de novo sequencing through MALDI TOFMS PSD (post source decay) are intensively studied for amino acid sequencing method of protein through mass spectrometry. For example, referred to are Charge Remote Fragmentation of Peptides Following Attachment of a Fixed Positive Charge: A Matrix-Assisted Laser Desorption/Ionization Postsource Decay Study, Pao-Chi Liao et al., J. Am, Soc. Mass Spectrum, 1997, 8, 501-509; A Method for High-Sensitivity Peptide Sequencing Using Postsource Decay Matrix-Assisted Laser Desorption Ionization, T. Keough et al., Proc. Natl. Acad. Sci. USA, Vol. 96, pp. 7131-7136 (1999); Peptide Sequencing of Charged Derivatives by Postsource Decay MALDI Mass Spectrometry, B. Spengler et al., International Journal of Mass Spectrome try and Ion Processes 169/170 (1997), 127-140. Using conventional mass spectrometers, however, no one has succeeded in completing a high-sensitivity method of detecting only the intended ion of various formable ion species (b, y, c, z, a, x).

### SUMMARY OF THE INVENTION

Given that situation, it is desired to develop a rapid and high-sensitivity method for determining the amino acid sequence of protein or peptide through mass spectrometry.

An object of the invention is to provide a novel metal complex useful for a reagent for determining the amino acid sequence of protein or peptide. Another object of the invention is to provide a reagent for determining the amino acid sequence of protein or peptide, which contains the novel metal complex. Still another object of the invention is to provide a method of using the novel metal complex for determining the amino acid sequence of protein or peptide.

The present inventors have assiduously studied and, as a result, have found that, by means of using a metal complex with a functional group capable of forming a covalent bond with the amino group of the N-terminal amino acid residue of protein or peptide or with the carboxyl group of the C-terminal amino acid residue of protein or peptide, as an amino acid sequencing reagent, thereby to obtain a metal complex derivative in which the functional group of the metal complex and the above amino group or the above carboxyl group of the protein or peptide has formed a covalent bond which is not cleaved in the stage of ionization in mass spectrometry, and analyzing the resulting metal complex derivative through mass spectrometry, the protein or peptide can be analyzed rapidly at high sensitivity. On the basis of this finding, we have reached the invention.

The invention includes the following:

(1) A metal complex which has a functional group capable of forming a covalent bond with an amino group of an N-terminal amino acid residue of protein or peptide or with a carboxyl group of a C-terminal amino acid residue of protein or peptide.

(2) The metal complex of above (1), which has a ligand with the functional group capable of forming the covalent bond with the amino group of the N-terminal amino acid residue of protein or peptide or with the carboxyl group of the C-terminal amino acid residue of protein or peptide.

(3) The metal complex of above (1) or (2), wherein a metal element thereof is selected from transition metals and typical metals.

(4) The metal complex of any of above (1) to (3), wherein a coordination number thereof is 2, 3, 4, 5 or 6.

(5) The metal complex of any of above (1) to (4), wherein a ligand thereof is a monodentate ligand or a polydentate ligand.

(6) The metal complex of any of above (1) to (5), wherein the covalent bond formed between the amino group of the N-terminal amino acid residue of protein or peptide or the carboxyl group of the C-terminal amino acid residue of protein or peptide and the functional group is not cleaved in a stage of ionization in mass spectrometry

(7) The metal complex of any of above (1) to (6), wherein the functional group capable of forming the covalent bond with the amino group of the N-terminal amino acid residue of protein or peptide is a functional group capable of forming the covalent bond through nucleophilic reaction with the amino group.

(8) The metal complex of any of above (1) to (7), wherein the functional group capable of forming the covalent bond with the amino group of the N-terminal amino acid residue of protein or peptide is —CO—OR₁, where R₁ represents H or an active ester-forming group.

(9) The metal complex of any of above (1) to (8), which is represented by the following general formula (I):

(L₂)mM(L₁) (I)

wherein M represents a transition metal; L₁ represents a ligand having a substituent: —CO—OR₁ (where R₁ represents H or an active ester-forming group) or —R₂—CO—OR₁ (where R₂ represents an arylene group or an alkylene group, R₁ represents H or an active ester-forming group); L₂ represents a ligand; m is a number of L₂, indicating 0, 1, 2, 3, 4 or 5.

(10) The metal complex of any of above (1) to (9), which is represented by the following general formula (II):

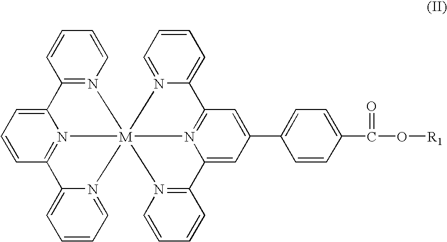

wherein M represents a transition metal; and R₁ represents H or an active ester-forming group represented by any of the following formula:

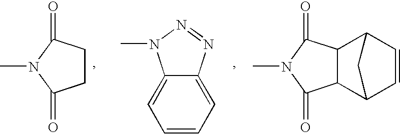

(11) The metal complex of any of above (1) to (6), wherein the functional group capable of forming the covalent bond with the carboxyl group of the C-terminal amino acid residue of protein or peptide is a functional group capable of forming the covalent bond through nucleophilic reaction with the carboxyl group.

(12) The metal complex of any of above (1) to (6) and (11), wherein the functional group capable of forming the covalent bond with the carboxyl group of the C-terminal amino acid residue of protein or peptide is —NH₂ or —NHNH₂.

(13) The metal complex of any of above (1) to (6), (11) and (12), which is represented by the following general formula (III):

(L₂)mM(L₃) (III)

wherein M represents a transition metal; L₃ represents a ligand having a substituent: —NH₂, —NHNH₂, —R₂—NH₂ or —R₂—NHNH₂ (where R₂ represents an arylene group or an alkylene group); L₂ represents a ligand; m is a number of L₂, indicating 0, 1, 2, 3, 4 or 5.

(14) A reagent for determining the amino acid sequence of protein or peptide, which comprises the metal complex of any of above (1) to (13).

(15) A method for determining the amino acid sequence of protein or peptide, which comprises using the metal complex of any of above (1) to (13).

(16) A method for determining the amino acid sequence of protein or peptide, which comprises

reacting the metal complex of any of above (1) to (13) with a protein or peptide (A) of which the amino acid sequence is to be determined, to form a metal complex derivative (B) where the covalent bond of the functional group of the metal complex with the amino group of the N-terminal amino acid residue of the protein or peptide (A) or with the carboxyl group of the C-terminal amino acid residue of protein or peptide is formed, and

analyzing the metal complex derivative (B) through mass spectrometry.

According to the present invention, there is provided the novel metal complex that is useful for a reagent for determining the amino acid sequence of protein or peptide, and provided the reagent that contains the novel metal complex for determining the amino acid sequence of protein or peptide.

Further, according to the present invention, there is provided the method of using the novel complex for determining the amino acid sequence of protein or peptide through mass spectrometry. The amino acid sequencing method of the present invention is extremely excellent in that it enables rapid and high-sensitivity sequencing.

### BRIEF DESCRIPTION OF THE DRAWINGS

FIG. 1 shows ESIMS charts obtained in Example.

FIG. 2 shows ESIMS charts obtained in Example.

FIG. 3 shows ESIMS charts obtained in Example.

FIG. 4 shows ESIMS charts obtained in Example.

### DETAILED DESCRIPTION OF THE INVENTION

The present invention relates to the metal complex having, in one molecule, one functional group capable of forming a covalent bond with the amino group of the N-terminal amino acid residue of protein or peptide or with the carboxyl group of the C-terminal amino acid residue of protein or peptide. In general, the functional group is in the ligand of the metal complex. For example, the functional group is in the ligand of the metal complex, via an arylene group such as phenylene group or an alkylene group and the like therein.

The metal element in the metal complex is selected from transition metals and typical metals. The transition metals include, for example, Sc, Ti, V, Cr, Mn, Fe, Co, Ni, Cu, Zr, Nb, Mo, Tc, Ru, Rh, Pd, Ag, Ta, W, Os, Ir, Pt, Au and the like. The typical metals include, for example, Zn, Al, As, Si, P and the like. Of those metals, preferred are Ru, Cr, Fe, Co, Ni, Cu, Rh, Pd, Os, Ir, Pt and the like.

The coordination number of the metal complex may be any of 2, 3, 4, 5 or 6, but is preferably 6 in view of the stability of the complex.

Further, the ligand in the metal complex may be any of a monodentate ligand or a polydentate ligand. The monodentate ligand includes amines (including cyclic amines such as imidazole), pyridine, oxo ligands such as carboxylic acids, and halogens and the like. Of the polydentate ligand, a bidentate ligand includes bipyridine, Schiff bases, phenanthrene, orthobenzoquinone derivatives, nucleic acid bases and the like. A tridentate ligand includes diethylenetriamine, terpyridine, Schiff bases, triazacycloalkanes, tetrakis(2′-aminoethyl)-1,2-diaminopropane, octaazabicyclo[6.6.6]eicosane and the like. A tetradentate ligand includes porphyrin and its derivatives, phthalocyanine and its derivatives, tetrazacycloalkanes and the like. A pentadentate ligand includes aminoalkyl-tetrazacycloalkanes and the like. A hexadentate ligand includes tri (aminoalkyl) triazacycloalkanes, 1,14-diamino-3,6,9,12-tetrazatetradecane and the like. Of those ligands, preferred are terpyridine, bipyridine, porphyrin and its derivatives (e.g., tetraphenylporphyrin), phthalocyanine and its derivatives and the like, as they are readily introduced the above-mentioned functional group.

Preferably in the invention, the covalent bond formed between the functional group and the amino group of the N-terminal amino acid residue of protein or peptide or the carboxyl group of the C-terminal amino acid residue of protein or peptide is not cleaved in the stage of ionization in mass spectrometry. If the covalent bond is cleaved in the stage of ionization, it is difficult to utilize in mass spectrometry in the amino acid sequencing method of the invention that will be mentioned hereinunder.

First described is the metal complex (the type 1) having a functional group capable of forming a covalent bond with the amino group of the N-terminal amino acid residue of protein or peptide.

In this aspect, the functional group of the metal complex is capable of forming a covalent bond due to a nucleophilic reaction with the amino group of the N-terminal amino acid residue of protein or peptide. For example, the functional group of the metal complex is represented by —CO—OR₁, where R₁ represents H or an active ester-forming group.

Accordingly, for example, the metal complex may be represented by the following general formula (I):

(L₂)mM(L₁) (I)

wherein M represents the above-mentioned transition metal; L₁ represents a ligand having a substituent: —CO—OR₁ (where R₁ has the same meaning as above) or —R₂—CO—OR₁ (where R₂ represents an arylene group or an alkylene group, R₁ has the same meaning as above); L₂ represents a ligand; m is a number of L₂, indicating 0, 1, 2, 3, 4 or 5. Example of the arylene group for R₂ is phenylene group; and the example of alkylene group is a lower alkylene group such as methylene, ethylene or propylene. In view of the easiness in nucleophilic reaction with the amino group, R₂ is preferably a phenylene group. Preferred examples of the metal are Ru, Cr, Fe, Co, Ni, Cu, Rh, Pd, Os, Ir, Pt and the like.

Preferably, the metal complex is represented by the following general formula (II):

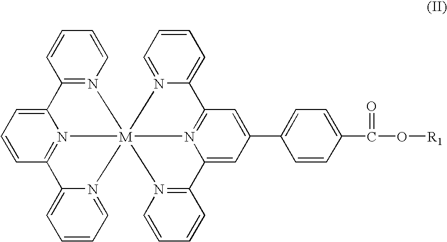

wherein M represents the above-mentioned transition metal; and R₁ represents H or an active ester-forming group mentioned below. The active ester-forming group may be any one capable of undergoing nucleophilic reaction with the amino group, and is therefore not limited to the following examples. The metal is preferably Ru, Os, Rh and the like.

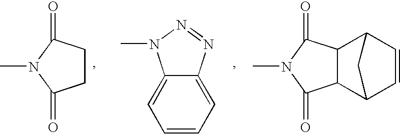

For the functional group of the metal complex, isocyanate and isothiocyanate groups may be taken into consideration. However, the covalent bond to be formed between these functional group and the amino group of the N-terminal amino acid residue of protein or peptide is readily cleaved in the stage of ionization in mass spectrometry, therefore, this case is unsuitable in utilizing for the amino acid sequencing method through mass spectrometry of the invention that is mentioned hereinunder.

The metal complex having the above functional group of the invention may be produced by first preparing a ligand having one functional group mentioned above, then coordinating the ligand, optionally along with any other ligand not having the above-mentioned functional group, to a metal. For example, in the case of the metal complex represented by formula (II); first prepared is 2,2′:6′, 2″-terpyridine with ap-carboxyphenyl group introduced into the 4-position. Then, the carboxyphenyl group-introduced 2,2′:6′,2″-terpyridine and 2,2′:6′,2″-terpyridine not having a functional group are coordinated to a metal M to obtain the carboxyphenyl group-having bis(terpyridine) metal complex (of formula (II) where R₁=H). For obtaining the metal complex of formula (II) where R₁ is an active ester-forming group, the metal complex with R₁=H obtained in the above may be condensed with a reagent of actively esterifying a carboxyl group (of a general formula R₁—OH, concretely, for example, N-hydroxysuccinimide) by the use of a condensing agent.

The metal complex of the invention may be identified through nuclear magnetic resonance spectrometry, visible or UV absorption spectrometry, mass spectrometry or the like.

Next described is the metal complex (the type 2) having a functional group capable of forming a covalent bond with the carboxyl group of the C-terminal amino acid residue of protein or peptide.

In this aspect, the functional group of the metal complex is capable of forming a covalent bond due to a nucleophilic reaction with the carboxyl group of the C-terminal amino acid residue of protein or peptide. For example, the functional group of the metal complex is represented by —NH₂ or —NHNH₂.

Accordingly, for example, the metal complex is represented by the following general formula (III):

(L₂)mM(L₃) (III)

wherein M represents the above-mentioned transition metal; L₃ represents a ligand having a substituent: —NH₂, —NHNH₂, —R₂—NH₂ or —R₂—NHNH₂ (where R₂ represents an arylene group or an alkylene group); L₂ represents a ligand; m is a number of L₂, indicating 0, 1, 2, 3, 4 or 5. Example of the arylene group for R₂ is phenylene group; and example of the alkylene group is a lower alkylene group such as methylene, ethylene or propylene group. In view of the easiness in nucleophilic reaction to the ester carbonyl group, R₂ is preferably a lower alkylene group. Preferred examples of the metal are Ru, Os, Rh and the like. Accordingly, preferred examples are metal complexes where L₃ is 2,2′:6′2″-terpyridine having aminoethyl group at 4-position, L₂ is 2,2′:6′,2″-terpyridine (m=1), and M is Ru, Os or Rh. Also preferred examples are metal complexes where L₃ is 2,2′:6′2″-terpyridine having amimophenyl group at 4-position, L₂ is 2,2′:6′,2″-terpyridine (m=1), and M is Ru, Os or Rh.

The metal complex of the type 2 having a functional group mentioned above of the invention may be produced and identified in the same manner as that for the metal complex of the type 1 mentioned above.

The metal complex having a functional mentioned above of the invention is extremely useful for a reagent for determining the amino acid sequence of protein or peptide through mass spectrometry. Accordingly, the invention relates to a method of using the metal complex having a functional group mentioned above, for determining the amino acid sequence of protein or peptide.

The amino acid sequencing method of the invention comprises reacting the metal complex of the type 1 having a functional group mentioned above of the invention with a protein or peptide (A) of which the amino acid sequence is to be determined, to thereby obtain a metal complex derivative (B) where the covalent bond of the functional group of the metal complex with the amino group of the N-terminal amino acid residue of the protein or peptide (A) is formed, followed by analyzing the obtained metal complex derivative (B) through mass spectrometry to thereby determine the amino acid sequence of the protein or peptide.

The amino acid sequencing method of the invention comprises reacting the metal complex of the type 2 having a functional group mentioned above of the invention with a protein or peptide (A) of which the amino acid sequence is to be determined, to thereby obtain a metal complex derivative (B) where the covalent bond of the functional group of the metal complex with the carboxyl group of the C-terminal amino acid residue of the protein or peptide (A) is formed, followed by analyzing the obtained metal complex derivative (B) through mass spectrometry to thereby determine the amino acid sequence of the protein or peptide.

The amino acid sequencing method with the metal complex of the type 1 is described in more detail. The following chemical reaction formula shows the case of using a ruthenium complex (1) of formula (II).

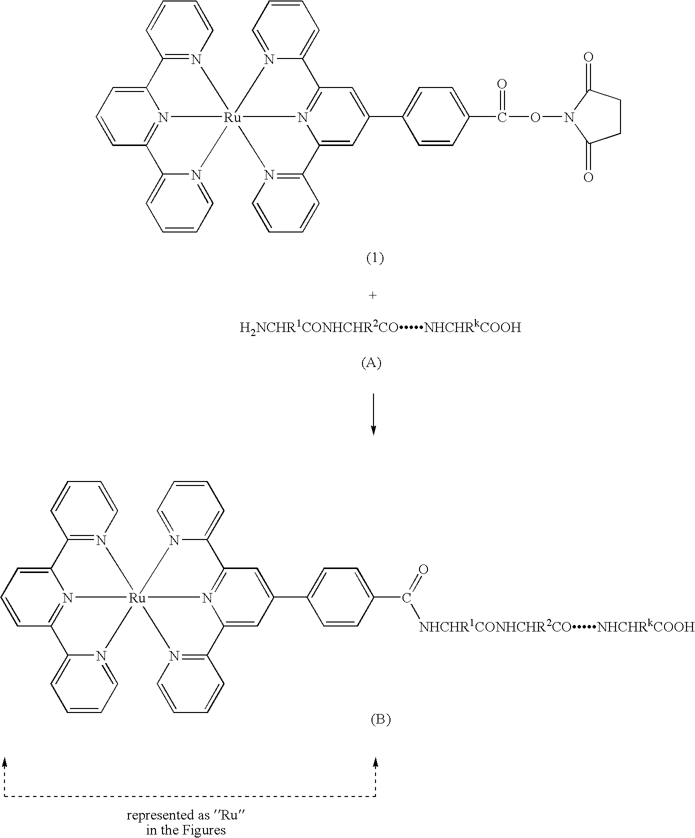

The ruthenium complex (1) is reacted with a protein or peptide (A) of which the amino acid sequence is to be determined, in a suitable solvent such as dimethylformamide to thereby obtain a ruthenium complex derivative (B) where the active ester group of the ruthenium complex (1) forms an amido bond with the amino group of the N-terminal amino acid residue of the protein or peptide (A). The reaction solution that contains the resulting ruthenium complex derivative (B) is diluted with a suitable diluent solvent such as methanol, and this is analyzed through mass spectrometry. Since the amido bond formed in the ruthenium complex derivative (B) is not cleaved in the stage of ionization and since the ionization efficiency of the ruthenium complex-containing ions is high, a-series and b-series ions are essentially observed. From the characteristic of the ruthenium atom isotope distribution, a-series and b-series ions are very easy to see.

The mass spectrometry is not specifically limited, for which, however, preferred is LCMS or MALDI-TOFMS. LCMS is liquid chromatographic mass spectrometry; and MALDI is matrix-assisted laser desorption/ionization; and TOFMS is time of flight mass spectrometry.

When the metal complex of the type 2 is used, its functional group such as —NH₂ or —NHNH₂ forms a covalent bond with the carboxyl group of the C-terminal amino acid residue of protein or peptide through nucleophilic reaction to give a ruthenium complex derivative (B). The reaction solution that contains the ruthenium complex derivative (B) is diluted with a suitable diluent solvent such as methanol, and this is analyzed through mass spectrometry. Since the covalent bond formed in the ruthenium complex derivative (B) is not cleaved in the stage of ionization and since the ionization efficiency of the ruthenium complex-containing ions is high, z-series and y-series ions are essentially observed. From the characteristic of the ruthenium atom isotope distribution, z-series and y-series ions are very easy to see.

The amino acid sequencing method of the invention is extremely rapid and highly sensitive (about 100 fmol), as compared with Edman reaction sequencing.

### EXAMPLES

The invention is described in more detail with reference to the following Examples, which, however, are not whatsoever intended to restrict the scope of the invention.

### Example 1

### Production of Metal Complex

In this Example, the ruthenium complex of formula (1) mentioned above chemical reaction formula was produced in the manner mentioned below. 2,2′:6′,2″-terpyridine is represented by tpy.

[(tpy)RuCl₃] was produced according to the method described in a reference, D. P. Sullivan et al., Inorg. Chem., 1980, 19, 1404-1407. 4′-(4-Carboxyphenyl)-2,2′:6′,2″-terpyridine (tpy-C₆H₄-COOH) was prepared according to the method described in a reference, G. D. Storrier et al., Inorg. Chim. Acta., 1999, 284, 76-84.

200 mg (0.45 mmols) of [(tpy)RuCl₃], 160 mg (0.45 mmols) of tpy-C₆H₄—COOH, and 1.0 ml (7.2 mmols) of triethylamine were refluxed for 18 hours in 100 ml of ethanol in an argon atmosphere. The resulting reaction liquid was concentrated under reduced pressure and dried to solid. Methanol was poured to it, and the methanol-soluble part was collected and purified through a column filled with Sephadex LH-24. The purified product was once dried to solid, and dissolved in water, and then an aqueous NH₄ PF₆ solution was added to the resulting aqueous solution. The deposit formed was collected through filtration, washed with water and dried to obtain [(tpy)Ru(tpy-C₆H₄—COOH)](PF₆)₂ (115 mg, yield 45%).

30 mg (0.03 mmols) of [(tpy)Ru(tpy-C₆H₄—COOH)](PF₆)₂ and 30 mg (0.26 mmols) of N-hydroxysuccinimide were dissolved in 3 ml of acetonitrile, and 0.1 ml (0.55 mmols) of WSCD was added to the resulting solution with cooling with ice, and stirred overnight. The resulting reaction liquid was concentrated under reduced pressure, and then ethyl acetate was added thereto. The deposit formed was collected through filtration, washed with ethyl acetate, and dried under reduced pressure to obtain [(tpy)Ru(tpy-C₆H₄—CO—NSu)](PF₆)₂ (30 mg, yield 90%).

WSCD is a water-soluble carbodiimide, 1-ethyl-3-(3-dimethylaminopropyl)carbodiimide. —NSu means a succinimido group.

Identification Data of [(tpy)Ru(tpy-C₆H₄—CO—NSu)](PF₆)₂:

¹H NMR(303K, dimethyl sulfoxide-d₆): δ 2.94 (brs, 4H, H_{SU}), 7.27 (m, 4H, H_{5A}, H_{5B}), 7.46 (d, 2H, J=5.6 Hz, H_{6A}), 7.54 (d, 2H, J=5.2 Hz, H_{6B}), 8.02 (t, 2H, J=8.0 Hz, H_{4A}), 8.08 (t, 2H, J=7.6 Hz, H_{4B}), 8.46 (d, 2H, J=8.4 Hz, H_{O}), 8.55 (t, 1H, J=8.0 Hz, H_{4′B}), 8.70 (d, 2H, J=8.8 Hz, Hₘ), 8.84 (d, 2H, J=8.4 Hz, H_{3B}), 9.11 (t, 2H, J=7.6 Hz, H_{3A}, H_{3′B}), 9.58 (s, 2H, H_{3′A})

ESI-MS m/z for C₄₁H₂₉N₇O₄Ru M²⁺ calcd. 392.4, found. 392.4, [M+PF₆]⁺ calcd. 929.7, found. 929.7

### Example 2

### Amino Acid Sequencing

This Example is to demonstrate amino acid sequencing with the Ru complex (1) produced in Example 1. The sample of amino acid tested herein is H-Gly-Gly-Tyr-Arg-OH.

In accordance with the chemical reaction formula mentioned above, the reagent, Ru complex (1) and H-Gly-Gly-Tyr-Arg-OH were dissolved in N,N′-dimethylformamide in an equivalent amount, and reacted with stirring overnight at room temperature.

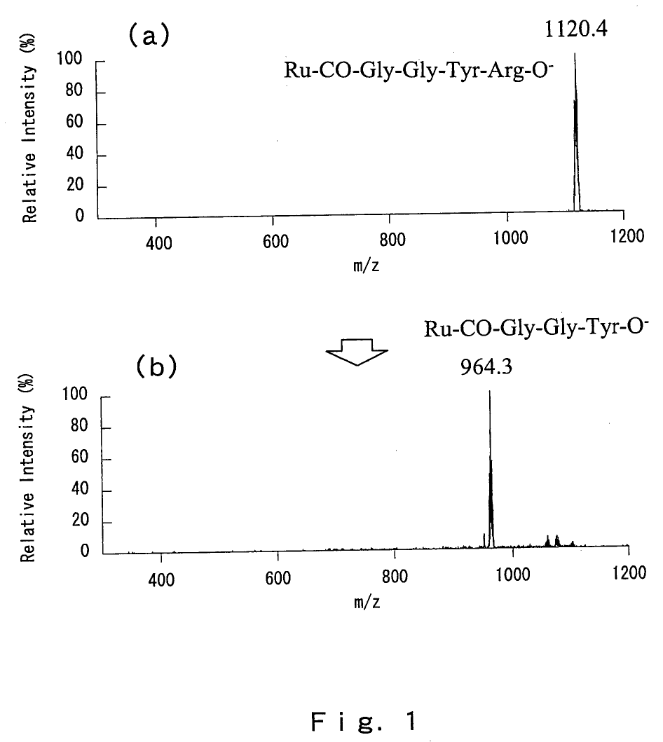

The reaction solution containing the Ru complex derivative of formula (B) was diluted with methanol, and analyzed through LCMS (ESIMS). FIGS. 1 to 4 show the LC-MS/MS data (ion trap) charts, in which the vertical axis indicates a relative intensity (%). In these figures and in the following description, the moiety containing metal and ligands comprising the phenylene group-containing ligand in the Ru complex derivative of formula (B) is represented by “Ru” for convenience sake.

In FIG. 1, (a) is an ESIMS chart of the Ru complex derivative (B) prepared in the above, Ru—CO-Gly-Gly-Tyr-Arg-OH; and (b) is a chart as a result of MS² with its peak as a parent ion.

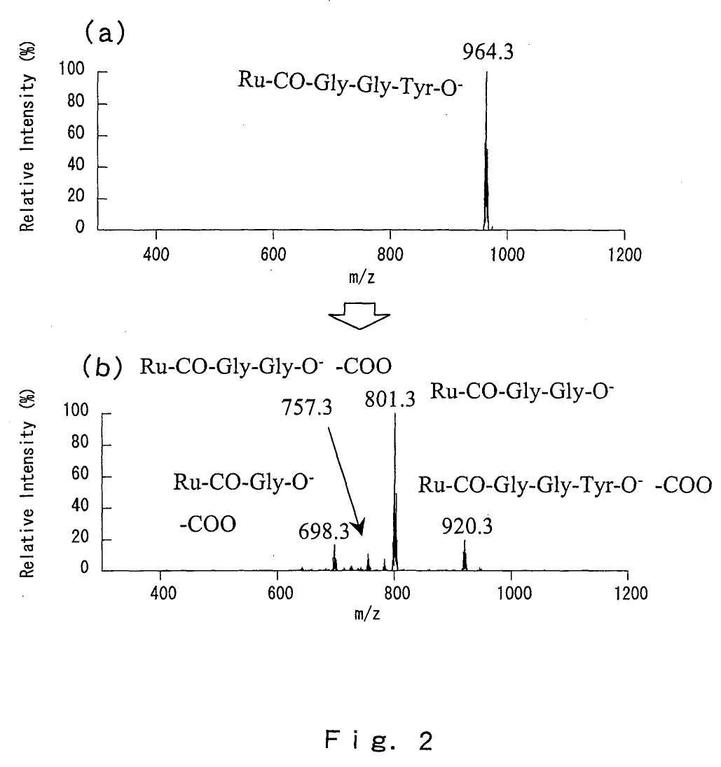

In FIG. 2, (a) is an MS² chart; and (b) is a chart as a result of MS³ with the MS² peak as a parent ion.

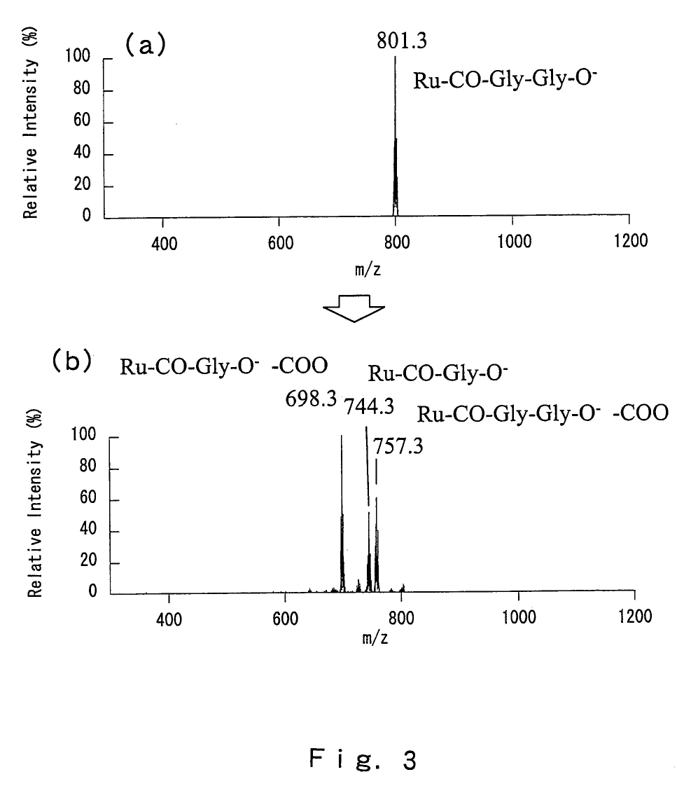

In FIG. 3, (a) is an MS³ chart; and (b) is a chart as a result of MS⁴ with the MS³ peak as a parent ion.

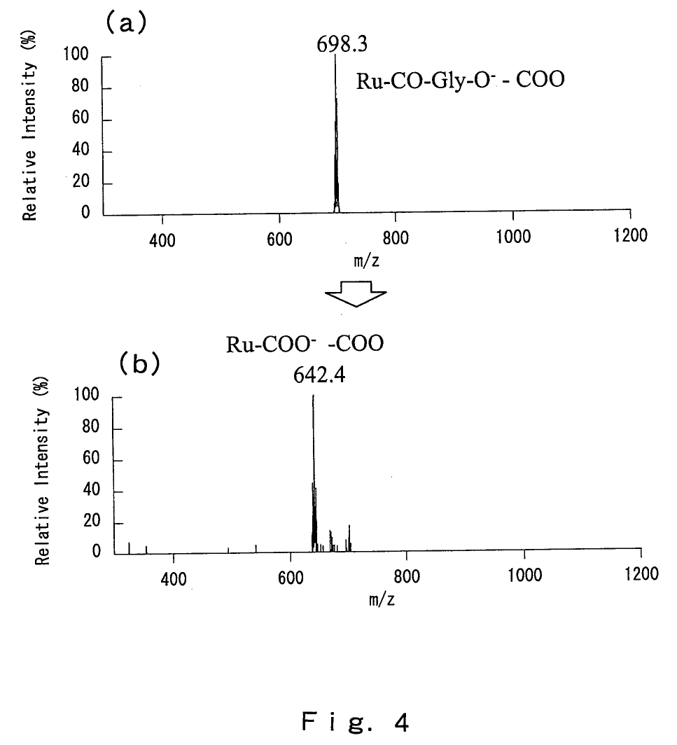

Similarly in FIG. 4, (a) is an MS⁴ chart; and (b) is a chart as a result of MS⁵ with the MS⁴ peak as a parent ion.

The data confirm that Ru complex (1) produced in Example 1 enables easy and high-sensitivity amino acid sequencing.

As this invention may be embodied in several forms without departing from the spirit or essential characteristics thereof, the foregoing examples are therefore only illustrative and should not be interpreted as restrictive, and all changes that fall within equivalence of claims are therefore intended to be embraced by the claims.

What is claimed is:

1. A metal complex which has a functional group capable of forming a covalent bond with an amino group of an N-terminal amino acid residue of protein or peptide or with a carboxyl group of a C-terminal amino acid residue of protein or peptide.

2. The metal complex according to claim 1, which has a ligand with the functional group capable of forming the covalent bond with the amino group of the N-terminal amino acid residue of protein or peptide or with the carboxyl group of the C-terminal amino acid residue of protein or peptide.

3. The metal complex according to claim 1, wherein a metal element thereof is selected from transition metals and typical metals.

4. The metal complex according to claim 1, wherein a coordination number thereof is 2, 3, 4, 5 or 6.

5. The metal complex according to claim 1, wherein a ligand thereof is a monodentate ligand or a polydentate ligand.

6. The metal complex according to claim 1, wherein the covalent bond formed between the amino group of the N-terminal amino acid residue of protein or peptide or the carboxyl group of the C-terminal amino acid residue of protein or peptide and the functional group is not cleaved in a stage of ionization in mass spectrometry.

7. The metal complex according to claim 1, wherein the functional group capable of forming the covalent bond with the amino group of the N-terminal amino acid residue of protein or peptide is a functional group capable of forming the covalent bond through nucleophilic reaction with the amino group.

8. The metal complex according to claim 1, wherein the functional group capable of forming the covalent bond with the amino group of the N-terminal amino acid residue of protein or peptide is —CO—OR₁, where R₁ represents H or an active ester-forming group.

9. The metal complex according to claim 1, which is represented by the following general formula (I):
(L₂)mM(L₁) (I)
wherein M represents a transition metal; L₁ represents a ligand having a substituent: —CO—OR₁ (where R₁ represents H or an active ester-forming group) or —R₂—CO—OR₁ (where R₂ represents an arylene group or an alkylene group, R₁ represents H or an active ester-forming group); L₂ represents a ligand; m is a number of L₂, indicating 0, 1, 2, 3, 4 or 5.

10. The metal complex according to claim 1, which is represented by the following general formula (II): 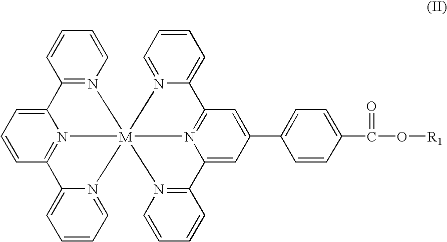
wherein M represents a transition metal; and R₁ represents H or an active ester-forming group represented by any of the following formula:

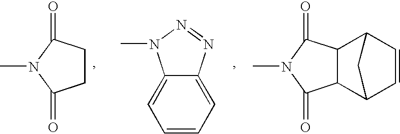

11. The metal complex according to claim 1, wherein the functional group capable of forming the covalent bond with the carboxyl group of the C-terminal amino acid residue of protein or peptide is a functional group capable of forming the covalent bond through nucleophilic reaction with the carboxyl group.

12. The metal complex according to 1, wherein the functional group capable of forming the covalent bond with the carboxyl group of the C-terminal amino acid residue of protein or peptide is —NH₂ or —NHNH₂.

13. The metal complex as claimed in claim 1, which is represented by the following general formula (III):
(L₂)mM(L₃) (III)
wherein M represents a transition metal; L₃ represents a ligand having a substituent: —NH₂, —NHNH₂, —R₂—NH₂ or —R₂—NHNH₂ (where R₂ represents an arylene group or an alkylene group); L₂ represents a ligand; m is a number of L₂, indicating 0, 1, 2, 3, 4 or 5.

14. A reagent for determining amino acid sequence of protein or peptide, which comprises the metal complex according to claim 1.

15. A method for determining amino acid sequence of protein or peptide, which comprises using the metal complex according to claim 1.

16. A method for determining amino acid sequence of protein or peptide, which comprises
reacting the metal complex according to claim 1 with a protein or peptide (A) of which the amino acid sequence is to be determined, to form a metal complex derivative (B) where the covalent bond of the functional group of the metal complex with the amino group of the N-terminal amino acid residue of the protein or peptide (A) or with the carboxyl group of the C-terminal amino acid residue of protein or peptide is formed, and
analyzing the metal complex derivative (B) through mass spectrometry.


SEQUENCE LISTING (2 sequences)

SEQ ID NO: 1 - protein, 4 aa, Artificial Sequence
  Description of Artificial Sequence synthetic peptide
GGYR

SEQ ID NO: 2 - protein, 4 aa, Artificial Sequence
  Description of Artificial Sequence synthetic peptide
GGYR